(19) United States
(12) Patent Application Publication
(10) Pub. No.: US 2017/0056255 A1
(43) Pub. Date: Mar. 2, 2017

(54) ABSORBENT ARTICLE WITH SIGNALS

(71) Applicant: The Procter & Gamble Company, Cincinnati, OH (US)

(72) Inventors: Theodore Cory Fites, Cincinnati, OH (US); Alizha Victoria SMITH, Wyoming, OH (US); John Andrew STRASEMEIER, Aurora, IN (US); Ann Cecilia TAPP, West Chester, OH (US)

(21) Appl. No.: 14/870,680

(22) Filed: Sep. 30, 2015

Related U.S. Application Data
(63) Continuation of application No. 62/210,005, filed on Aug. 26, 2015.
(60) Provisional application No. 62/210,014, filed on Aug. 26, 2015.
(60) Provisional application No. 62/210,020, filed on Aug. 26, 2015.
(60) Provisional application No. 62/210,100, filed on Aug. 26, 2015.

(51) Int. Cl. A61F 13/49; A61F 13/53; A61F 13/494; A61F 13/84

(52) U.S. Cl. CPC A61F 13/49; A61F 13/84; A61F 13/53; A61F 13/49466; A61F 2013/8497; A61F 2013/530905; A61F 2013/530226; A61F 2013/530379; A61F 2013/49092; A61F 2013/4948

(57) ABSTRACT

An absorbent article comprising a topsheet, a backsheet, an absorbent core disposed between the topsheet and the backsheet, a longitudinal central axis and a transverse central axis, and a pattern visible through the topsheet. The pattern includes a first design element that repeats to form a primary motif, a second design element that repeats to form a secondary motif, and blue.

### CROSS REFERENCE TO RELATED APPLICATIONS

This application claims priority under 35 U.S.C. §119(e) to Provisional Patent Application Ser. No. 62/210,005 (P&G 13971PQ), 62/210,014 (P&G 13972PQ), 62/210,020 (P&G 13973PQ), 62/210,100 (P&G 13999PQ), all of which were filed on Aug. 26, 2015. The entire disclosures of all of the above-referenced U.S. Provisional patent applications are fully incorporated herein by reference.

### FIELD OF THE INVENTION

The present invention relates to absorbent articles comprising indicia, especially indicia comprising printed adhesive signals.

### BACKGROUND OF THE INVENTION

Absorbent articles for receiving and retaining bodily discharges such as urine or feces are well known in the art. Examples of these include disposable diapers, training pants and adult incontinence articles. Typically, disposable diapers comprise a liquid pervious topsheet that faces the wearer's body, a liquid impervious backsheet that faces the wearer's clothing and an absorbent core interposed between the liquid pervious topsheet and the backsheet.

An important component of absorbent articles is the absorbent core. The absorbent core typically includes absorbent polymer material, which ensures that large amounts of bodily fluids, e.g. urine, can be absorbed by the absorbent article during its use and be locked away, thus providing low rewet and good skin dryness.

Traditionally, the superabsorbent polymer material is incorporated into the absorbent core structure with pulp, i.e. cellulose fibers. To make the absorbent core structures thinner, it has been proposed to reduce or eliminate these cellulose fibers from the absorbent core structures.

However, it was found that some absorbent core structures with reduced cellulose fiber levels may have an increased stiffness when loaded, especially in those regions which comprise most of the absorbent capacity of the absorbent article, such as the front region and crotch region of the article. It has been found that by providing specific permanent channels that are free of absorbent polymer material, improved liquid transport is achieved and, hence, faster acquisition and more efficient liquid absorbency over the whole absorbent layer. Even though less absorbent material may be used, improved performance can be achieved. Further, by immobilizing the absorbent material or channels by using adhesive, the channels remain during the use (e.g. when friction is applied to the absorbent layer, or when the absorbent layer is wetted and the absorbent material expands). Furthermore, it has been found that providing such channels results in increased flexibility in the article without undermining the article's performance during use.

Regardless of the construction of the absorbent core, there is a continued need to communicate the performance capability (e.g., absorbency) of the core as well as the performance, softness, comfort and fit of the disposable article as a whole. Particularly, as the functionality of the absorbent core and article are changed to improve (e.g., absorbency, fit, or reduced cost), the appearance of the article may likewise change. Consumers and/or wearers of the articles may not appreciate that the changes are not adversely affecting product performance. Similarly, consumers and/or wearers may not readily identify improvements to articles without visual cues from manufacturers. Therefore, there is a continued need to provide signals to the consumer and/or wearer that an absorbent article will perform as desired. Further, there is a need to provide optimal signals while maintaining excellent process capability and technical product performance.

### SUMMARY OF THE INVENTION

In an embodiment, an absorbent article includes a topsheet, a backsheet, an absorbent core disposed between the topsheet and the backsheet, a longitudinal central axis, a transverse central axis, and a pattern visible through the topsheet. The pattern comprises a first design element that repeats to form a primary motif and a second design element that repeats to form a secondary motif. The pattern further comprises blue.

In another embodiment, an absorbent article includes a topsheet, a backsheet, an absorbent core disposed between the topsheet and the backsheet, a longitudinal axis and a transverse axis. The absorbent core comprises a pair of channels, symmetrically disposed about the longitudinal axis. The article further comprises a pattern visible through the topsheet, wherein the pattern comprises a first design element that repeats to form a primary motif, a second design element that repeats to form a secondary motif, a channel element and blue.

### BRIEF DESCRIPTION OF THE DRAWINGS

FIG. 1 is a plan view of an absorbent article in accordance with one non-limiting embodiment;

FIG. 2 is a perspective view of an absorbent layer in accordance with one non-limiting embodiment;

FIG. 3 is a cross sectional view of an absorbent core in accordance with one non-limiting embodiment;

FIGS. 4-12 are plan views of example indicia for use in absorbent articles of the present disclosure; and

FIG. 13 is a plan view of exemplary backsheet graphics.

### DETAILED DESCRIPTION OF THE INVENTION

### Definitions

“Absorbent article” refers to a device that absorbs and contains body exudates, and, more specifically, refers to devices that are placed against or in proximity to the body of the wearer to absorb and contain the various exudates discharged from the body. Absorbent articles may include adult and infant diaper, including pants, such as infant training pants and adult incontinence undergarments, and feminine hygiene products, such as sanitary napkins and panty-liners and adult incontinent pads, and breast pads, care mats, bibs, wound dressing products, and the like. Absorbent articles may further include floor cleaning articles, food industry articles, and the like. As used herein, the term “body fluids” or “body exudates” includes, but is not limited to, urine, blood, vaginal discharges, breast milk, sweat and fecal matter.

As used herein “diaper” refers to devices which are intended to be placed against the skin of a wearer to absorb and contain the various exudates discharged from the body. Diapers are generally worn by infants and incontinent persons about the lower torso so as to encircle the waist and legs of the wearer. Examples of diapers include infant or adult diapers and pant-like diapers such as training pants. “Training pant”, as used herein, refers to disposable garments having a waist opening and leg openings designed for infant or adult wearers. A pant may be placed in position on the wearer by inserting the wearer's legs into the leg openings and sliding the pant into position about a wearer's lower torso. A pant may be pre-formed by any suitable technique including, but not limited to, joining together portions of the article using refastenable and/or non-refastenable bonds (e.g., seam, weld, adhesive, cohesive bond, fastener, etc.). A pant may be pre-formed anywhere along the circumference of the article (e.g., side fastened, front waist fastened).

“Disposable” is used herein to describe articles that are generally not intended to be laundered or otherwise restored or reused (i.e., they are intended to be discarded after a single use and, may be recycled, composted or otherwise disposed of in an environmentally compatible manner).

“Visible” as used herein means capable of being perceived by the unaided human eye.

“Diagonal” as used herein means an item disposed about ±10°-80°, or about ±25°-70°, or about ±30°-60°, or about ±45° with respect to longitudinal central axis of the core and/or the longitudinal axis of the article, disclosing for each range every 10° interval therebetween. Where a motif, pattern and/or plurality of elements are “diagonally disposed”, it is meant that a diagonal imaginary line intersects that the same location (e.g., the center) of like elements (e.g., two or more first elements, two or more second elements) within the motif, pattern, or plurality of elements.

“Pattern” as used herein means a decorative or distinctive design, not necessarily repeating or imitative, including but not limited to the following: marbled, check, mottled, veined, clustered, geometric, spotted, helical, swirl, arrayed, variegated, textured, spiral, cycle, contoured, laced, tessellated, starburst, lobed, lightning, blocks, textured, pleated, cupped, concave, convex, braided, tapered, and combinations thereof. In some embodiments, the pattern includes one or more repeating design elements.

“Design element” as used herein means a shape or combination of shapes that visually create a distinct and discrete component, regardless of the size or orientation of the component. A design element may be present in one or more motifs.

The present invention provides absorbent articles comprising absorbent cores and signals that communicate the absorbency, fit, comfort and/or other aspects of performance of the article. In some embodiments, the invention relates to absorbent cores comprising channels and printed signals that communicate the channels, such as those disclosed in U.S. patent application Ser. No. 14/077,355 filed on 12 Nov. 2013 and U.S. patent Ser. No. 14/487,115 filed on 16 Sep. 2014 (Attorney Docket Nos. 12661MQ and 13064M respectively). In certain embodiments, the present invention also relates to absorbent articles with channels disclosed in U.S. application Ser. Nos. 13/491,642, 13/491,643, 13/491,644, and 13/491,648, all filed Jun. 10, 2011.

Absorbent Articles

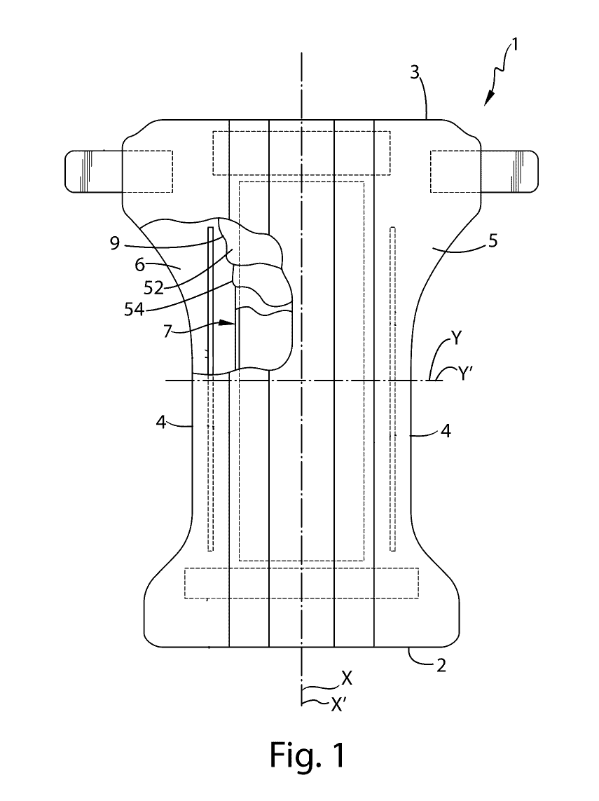

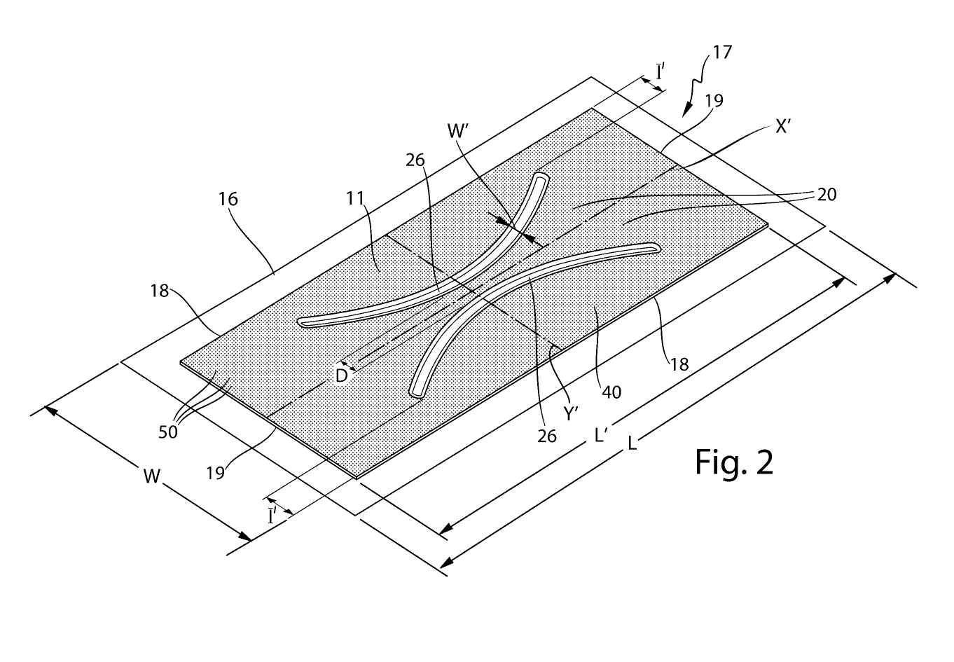

FIG. 1 is a plan view of an exemplary, non-limiting embodiment of an absorbent article 1 of the present invention in a flat, uncontracted state. The body-facing surface of the absorbent article 1 is facing the viewer. The absorbent article 1 includes a longitudinal axis X and a transverse axis Y, a front waist edge 2 and a back waist edge 3 and two longitudinal edges 4 extending between the front and back waist edges. The article may comprise further a topsheet 5, a backsheet 6, and an absorbent core 7 between the topsheet 5 and the backsheet 6. The absorbent article may also comprise an acquisition and/or distribution system (“ADS”) 9, which will be further detailed below. In addition, the article may comprise one or more side flaps or cuffs.

The topsheet 5 may be positioned at least in partial contact or close proximity to a wearer. Suitable topsheets 4 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Generally, at least a portion of the topsheet 5 is liquid pervious. The topsheet 5 may be apertured. An example of topsheet comprises a web of spunbond polypropylene fibers. Typical diaper topsheets have a basis weight of from about 10 to about 28 gsm, in particular between from about 12 to about 18 gsm but other basis weights are possible. Suitable formed film topsheets are for example described in U.S. Pat. Nos. 3,929,135; 4,324,246; 4,342,314; 4,463,045; and 5,006,394. Other suitable topsheets may be made in accordance with U.S. Pat. Nos. 4,609,518; 4,629,643 and U.S. patent application Ser. No. 14/634,928.

The backsheet may be liquid impervious. In some embodiments, the liquid impervious backsheet comprises a thin plastic film such as a thermoplastic film having a thickness of about 0.01 mm to about 0.05 mm. Suitable backsheet materials comprise typically breathable material, which permit vapors to escape from the article while still preventing exudates from passing through the backsheet. Suitable backsheet films include those manufactured by Tredegar Industries Inc. of Terre Haute, Ind. and sold under the trade names X15306, X10962 and X10964. A typical backsheet comprises a liquid-impermeable film, which may be joined with a softer non-woven layer on its surface.

The absorbent core will be discussed in more detail below.

The absorbent article may comprise an ADS 9. One function of the ADS is to quickly acquire one or more of the fluids and distribute them to the absorbent core in an efficient manner. The ADS may comprise one, two or more layers, which may form a unitary layer or may remain as discrete layers which may be attached to each other. In an example, the ADS may comprise two layers: a distribution layer 54 and an acquisition layer 52 disposed between the absorbent core and the topsheet, but the present disclosure is not so limited.

The ADS 9 may not comprise superabsorbent material (“SAP”) 50 as this may slow the acquisition and distribution of the fluids. Suitable ADS are described in WO 2000/59430 (Daley), WO 95/10996 (Richards), U.S. Pat. No. 5,700,254 (McDowall), and WO 02/067809 (Graef), for example.

In one example, the ADS 9 may not be provided, or only one layer of the ADS may be provided, such as the distribution layer 54 only or the acquisition layer 52 only.

The distribution layer 54 may comprise the cross-linked cellulose fibers as well as other fibers, but this layer may advantageously comprise at least 50%, or 60%, or 70%, or 80%, or 90%, or even up to 100%, by weight of the layer, of cross-linked cellulose fibers (including the cross-linking agents). Examples of such mixed layer of cross-linked cellulose fibers may comprise about 70% by weight of chemically cross-linked cellulose fibers, about 10% by weight polyester (PET) fibers, and about 20% by weight untreated pulp fibers. In another example, the layer of cross-linked cellulose fibers may comprise about 70% by weight chemically cross-linked cellulose fibers, about 20% by weight lyocell fibers, and about 10% by weight PET fibers. In still another example, the layer may comprise about 68% by weight chemically cross-linked cellulose fibers, about 16% by weight untreated pulp fibers, and about 16% by weight PET fibers. In yet another example, the layer of cross-linked cellulose fibers may comprise from about 90 to about 100% by weight chemically cross-linked cellulose fibers.

The acquisition layer 52 may be disposed between the distribution layer 54 and the topsheet 5. The acquisition layer 52 may be or may comprise a nonwoven material, such as a hydrophilic SMS or SMMS material, comprising a spunbonded, a melt-blown and a further spunbonded layer or alternatively a carded staple fiber chemical-bonded nonwoven. The nonwoven material may be latex bonded.

A further acquisition layer may be used in addition to a first acquisition layer described above. For example, a tissue layer may be placed between the first acquisition layer and the distribution layer. The tissue may have enhanced capillarity distribution properties compared to the acquisition layer described above.

The absorbent article may comprise a pair of partially upstanding barrier leg cuffs and/or elasticized gasketing cuffs. Nonlimiting examples of suitable cuffs are disclosed in U.S. Pat. Nos. 3,860,003; 4,808,178; 4,909,803; 4,695,278 and 4,795,454. All or a portion of the barrier leg and/or gasketing cuffs may be treated with a lotion.

The article may comprise side panels, or so-called ear panels. The article may comprise fastening means, to fasten the front and back. Fastening systems may comprise fastening tabs and landing zones, wherein the fastening tabs are attached or joined to the back region of the article and the landing zones are part of the front region of the article.

The absorbent article may also comprise other typical components, which are not represented, such as a back elastic waist feature, a front elastic waist feature, transverse barrier cuff(s), ears, fasteners, a lotion application, etc.

### Absorbent Core

As used herein, the term “absorbent core” refers to the component of the absorbent article having the most absorbent capacity and comprising an absorbent material and a core wrap or core bag (used interchangeably herein) enclosing the absorbent material 50. The term “absorbent core” does not include the acquisition and/or distribution system or any other components of the article which are not either integral part of the core wrap 10 or placed within the core wrap 10. The absorbent core 7 may comprise, consist essentially of, or consist of, a core wrap 10, an absorbent material (e.g., superabsorbent polymers) 50, and optionally one or more adhesives.

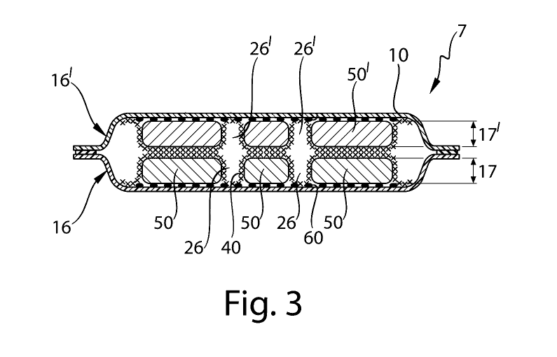

The absorbent core 7 comprises an absorbent layer 17, having a supporting sheet 16 with of absorbent material 50 disposed thereon as can been seen in FIGS. 2-3. The supporting sheet 16 may form the core wrap 10 or a portion of the core wrap 10.

By “absorbent material,” it is meant a material which has some absorbency property or liquid retaining properties, such as superabsorbent material (“SAP”), cellulosic fibers as well as synthetic fibers. Typically, glues used in making absorbent cores have no or little absorbency properties and are not considered as absorbent material. The SAP content may be higher than 80%, for example at least 85%, at least 90%, at least 95%, at least 99%, and even up to and including 100% of the weight of the absorbent material 50 contained within the core wrap 10. This provides a relatively thin core compared to a conventional core typically comprising between 40-60% SAP and high content of cellulose fibers. However, conventional cores are also within the scope of the present disclosure. The absorbent material 50 may comprise less than 15% weight percent or less than 10% weight percent of natural, cellulosic, or synthetic fibers, less than 5% weight percent, less than 3% weight percent, less than 2% weight percent, less than 1% weight percent, or may even be substantially free of natural, cellulosic, and/or synthetic fibers. The core wrap 10 is not considered as absorbent material 50 for the purpose of assessing the percentage of SAP in the absorbent core 7. The core 7 may also contain airfelt or cellulosic fibers with or without SAP.

The absorbent layer 17 comprises a longitudinal dimension and average length L extending in the longitudinal dimension. The layer 17 further comprises a transverse dimension and average width W extending in the transverse dimension. The layer 17 has a front region, being in use towards the front edge 2, and a back region, being in use towards the back edge 3, and therein between a crotch region, each extending the full transverse width of the layer, and each having ⅓ of the average length of the layer. The layer 17 possesses a central longitudinal axis X′, a central transverse axis Y′ perpendicular to said central longitudinal axis X′. The longitudinal axis and transverse axis may overlay the longitudinal and transverse axes of the article respectively. The layer further comprises a pair of opposing longitudinal side edges 18 extending in the longitudinal dimension of the structure or layer and a pair of opposing transverse edges 19 (e.g. front transverse edge being in use towards the front edge of the article, and a back transverse edge being in use towards the back edge of the article). The longitudinal side edges 18 and/or transverse edges 19 may be parallel to the central longitudinal axis X′ and/or central transverse axis Y′ respectively or one or more may be curvilinear, and for instance provide for a narrower transverse dimension in the crotch region. Typically the longitudinal side edges 18 are mirror images of one another in the longitudinal dimension.

The absorbent material 50 may be distributed in higher amount towards the front transverse edge 19 than towards the back transverse edge 19 as more absorbency may be required at the front in particular absorbent articles.

The central longitudinal axis X′ of the absorbent layer 17 delimits first and second longitudinal side portions 20 of the absorbent layer 17. Each of said longitudinal side portions is thus present in said front region, crotch region and back region. In some embodiments herein, said longitudinal portions 20 of the absorbent layer 17 are mirror images of one another in the longitudinal dimension.

In one nonlimiting example, the absorbent layer 17 comprises one or more longitudinally extending channels 26 shown in FIG. 2 and described in more detail below. Said channels 26 may be substantially free of absorbent material 50. In an alternative nonlimiting example, the absorbent layer 17 does not comprise channels 26.

As for example shown in FIG. 3, one or more adhesive materials 40, 60 may be present in the core. In one embodiment, a first adhesive material 40 may be applied on said absorbent layer 17 to immobilize the absorbent material 50 on the supporting sheet 16. The first adhesive material 40 may comprise a thermoplastic adhesive material. In one embodiment, the first adhesive material comprises a fibrous adhesive, as described herein after. In some embodiments, the first adhesive material 40 may be applied continuously over the absorbent layer 17, hence over the absorbent material 50 and in the channels 26 if any, to immobilize the absorbent layer and to optionally also adhere the supporting sheet in said channel.

In addition, or alternatively, the core may comprise a second adhesive material 60 which may be present to the supporting sheet 16 before the application of the absorbent material in order to enhance adhesion of the absorbent material as well as adhesion of the first adhesive material 40 to the supporting sheet 16. The second adhesive 60 may also aid in immobilizing the absorbent material 50 and may comprise the same thermoplastic adhesive material as described hereinabove or may also comprise other adhesives including but not limited to sprayable hot melt adhesives, such as H.B. Fuller Co. (St. Paul, Minn.) Product No. HL-1620-B. The second adhesive material may, for example, be applied to portions of said supporting sheet 16 that are to coincide with channels 26, so that in said channels the supporting sheet can be bonded with said adhesive to the walls of the channel, or part thereof or to a further material, as described below. The second adhesive material may be, for example, a hotmelt adhesive applied by printing, slot coating or spraying. In some embodiments, the first and second adhesive material may be the same type of adhesive, the difference between the first and second adhesive thus being the location where it is applied.

In some embodiments, part of the supporting sheet 16 may be folded over the absorbent layer 17 and then sealed along the peripheral edges, to enclose the absorbent layer 17, forming the core wrap 10. In other embodiments, the core wrap 10 is comprised of two supporting sheets 16, 16′ joined together to surround the absorbent material and said adhesive materials.

In some embodiments, the core 7 further comprises a second absorbent layer 17′. The second absorbent layer 17′ may comprise a second supporting sheet 16′ and absorbent material disposed thereon. The second absorbent layer 17′ may or may not comprise channels 26′ and/or adhesive 40, 60. The second absorbent layer 17′ may be identical to the first absorbent layer 17 or different (e.g., having different channels, different number of channels, different adhesive, different adhesive application or combinations thereof).

### Channels

In some embodiments (illustrated in FIGS. 2 and 3), the absorbent layer 17 may comprise a first channel 26 and a second channel 26 that are substantially free of (e.g., free of) superabsorbent polymer material 50. Other materials may be present in said channels 26, for example said one or more adhesive materials 40, 60. It should be understood that, accidentally, a small, negligible amount of superabsorbent polymer particles may be present in the channel, which does not contribute to the overall functionality. When the absorbent layer 17 comprises cellulosic or cellulose, in some embodiments, the first and second channels 26 are also free of such cellulosic/cellulose material. The channels 26 may extend through the thickness (height) of the absorbent layer 17.

The first channel 26 may be present in said first longitudinal side portion 20 of the absorbent layer 17 and the second channel 26 may be present in said second longitudinal side portion 20 of the absorbent layer 17.

The first and second channel 26 can each extend substantially longitudinally, which means typically that each channel 26 extends more in the longitudinal dimension than in the transverse dimension, and typically at least twice as much in the longitudinal dimension than in the transverse dimension. Thus, this includes channels 26 that are substantially parallel to the longitudinal axis X′ of said absorbent layer 17; and this includes channels 26 that may be curved, provided the radius of curvature is typically at least equal to the average transverse dimension of the absorbent layer (optionally at least 1.5 or at least 2.0 times this average transverse dimension); and this includes channels 26 that are straight but under an angle of from about 5° to about 30°, or up to about 20°, or up to about 10° with a line parallel to the longitudinal axis X′. This may also include channels with an angle therein, provided said angle between two parts of a channel is at least 120°, at least 150°; and in any of these cases, provided the longitudinal extension of the channel is more than the transverse extension.

In some embodiments, there may be no completely or substantially transverse channels present in at least said crotch region, or no such channels at all.

Each of said first and second channels 26 may have an average width W′ that is least 4% of the average width W of said absorbent layer 17, or for example W′ is at least 7% of W; and/or for example and up to 25% of W, or up to 15% of W; and/or for example at least 5 mm; and for example up to 25 mm, or for example up to 15 mm.

Each of said first and second channels 26 may have an average length L′ which may for example be up to 80% of the average length L of said absorbent layer 17. If the channels 26 are only in the front region, or only in the crotch region, or only in the back region, L′ is for example up to 25% of L, or up to 20% of L, and/or L′ is for example at least 5% of L, or at least 10% of L; and/or L′ is for example at least 10 mm, or at least 20 mm. If the channels 26 extend in said crotch region and front region, and optionally the back region, L′ is for example up to 80% of L, or up to 70% of L, and/or L′ is for example at least 40% of L, or at least 50% of L. In case the channel is not parallel to the longitudinal axis, the length L′ of the channel is the length as measured by projection against the longitudinal axis.

The channels 26 may typically be so-called “permanent” channels 26. By permanent, it is meant that the integrity of the channels 26 is at least partially maintained both in the dry state and in the wet state, including during friction by the wearer thereon. Permanent channels 26 may be obtained by provision of one or more adhesive material that immobilize said absorbent material 50, and/or said channels 26, and/or that immobilize said supporting sheet 16 into said channels 26, or part thereof. The absorbent core 7 may comprise permanent channels 26 formed by bonding of the first supporting sheet 16 and second supporting sheet 16′ through the channels 26. Typically, glue may be used to bond both supporting sheets through the channel, but it is possible to bond via other known means, for example ultrasonic bonding, or heat bonding. The supporting layers can be continuously bonded or intermittently bonded along the channels.

In some embodiments, said one or more adhesive materials 40, 60 are at least present in channels 26, for example at least said first adhesive material 40, or both said first and second adhesive material 40, 60. The adhesive material may thus be present on the longitudinal walls of the channels 26 (extending the height of the absorbent layer 17 and the length thereof). The supporting sheet 16 material may fold into said channels 26, or part thereof, creating undulations into said channels or part thereof, and said undulations may be fixed to said walls (or part thereof) to ensure the channels 26 are maintained (at least partially) during use.

Without being bound by theory, it is believed that permanent channels provide for fast liquid acquisition which reduces risk of leakages. The permanent channels help to avoid saturation of the absorbent layer in the region of fluid discharge (such saturation increases the risk of leakages). Furthermore, in contrast to what would be expected, while the overall amount of superabsorbent polymer material in the absorbent layer is reduced (by providing channels free of such material), the fluid handling properties of the absorbent layer is improved. Permanent channels also have the further advantage that in wet state, the absorbent material cannot move within the core and remains in its intended position, thus providing better fit and fluid absorption.

The channels 26 may comprise or consist of a pair of channels symmetrically arranged relative to the longitudinal axis X of the article and/or the longitudinal axis X′ of the absorbent layer. The channels 26 may be straight. The channels may be contoured as shown in FIG. 2; for example, they may be concave towards the longitudinal axis X′. The first and second channels 26 may be mirror images of one another with respect to the central longitudinal axis X′ of the absorbent layer 17. The channels 26, in particular when present as one or more symmetrical pair(s) relative to the longitudinal axis X′, may be spaced apart from one another over their whole longitudinal dimension. In one embodiment, the smallest spacing distance D (the distance between the innermost lines of the paired channels) may be at least 5% of average transverse dimension W of layer, or for example at least 10% of W, or at least 15% of W. In nonlimiting examples, the smallest spacing distance D may be for example at least 5 mm, or at least 10 mm, or at least 16 mm.

In some embodiments, there is no channel 26 that coincides with said longitudinal axis X′ of said absorbent layer 17.

Furthermore, in order to reduce the risk of fluid leakages, the longitudinal channels 26 typically do not extend up to any of the transverse edges 19 and/or longitudinal edges 18 of the absorbent layer 17. Typically, the smallest distance I′ between a channel 26 and the nearest longitudinal edge 18 corresponds to at least 5% of W, or for example to at least 10% of W. In some embodiments, the distance is for example at least 10 mm. The smallest distance between a channel and the nearest transverse edge 19 of the absorbent layer 17 may for example be at least 5% of the average length L of the layer.

The absorbent layer 17 may comprise only two channels 26, for example only in the front region, or for example in the central (crotch) region, and optionally extending into the front and/or back region, such as shown FIG. 2.

The absorbent layer 17 may comprise more than two of such channels 26, for example at least 4, or at least 5 or at least 6. Some or all of these may be substantially parallel to one another, for example being all straight and completely longitudinally, and/or two or more or all may be mirror images of one another about the longitudinal axis, or two or more may be curved or angled and for example mirror images of one another about the longitudinal axis X′, and two or more may be differently curved or straight, and for example mirror images of one another in the longitudinal dimension.

In one nonlimiting example, the front region of the absorbent layer 17 may comprise two or more channels 26 that are typically mirror images of one another about the longitudinal axis X′, and the crotch region may comprise two or more channels 26, that are typically mirror images of one another about the longitudinal axis X′, and the latter may optionally extend into the front region and/or back region, with any of applicable dimensions and other characteristics described above. Optionally, further channels may be present in the back region.

The first and second channels 26, and optionally further channels 26, may be positioned in said absorbent layer 17 such that there is a central longitudinal strip, coinciding with said longitudinal axis X′, which is free of any channels 26. Absorbent material 50 may be substantially continuously present in said strip. For example, said strip may have a minimum width D (corresponding to the distance between the innermost lines of two channels) of at least 5% of W, or at least 10% of W.

In some embodiments, in said central longitudinal strip between two neighboring channels 26, the average basis weight of absorbent material 50, or of said superabsorbent polymer material, is at least 350 grams per m², and for example up to 1000 grams per m², or for example from 450 grams per m², and for example up to 750 grams per m².

In some embodiments, adjacent each first and second channel 26, and optionally adjacent said further channels, said absorbent material 50 is substantially continuously present.

In embodiments where the core 7 comprises a first and second absorbent layer 17, 17′, each having channels 26, 26′, at least some of the channels 26 of the first absorbent layer 17 may coincide and overlap with at least some of the channels 26′ of the second absorbent layer 17′ (e.g., completely coincide and/or overlap, only partially coincide and/or overlap, and/or no overlap or coinciding of some channels 26, 26′). In some embodiments, the absorbent layers 17, 17′ are about identical to one another, and the channels 26 of one layer substantially completely coincide and overlap the channels 26 of another layer. One or more of the channels 26 of one absorbent layer 17 may substantially superpose the channels 26′ of the adjacent absorbent layer. The resulting absorbent core 7 is then a laminate of absorbent layers with channels 26, 26′, wherein the channels 26, 26′ extend substantially through the thickness of the absorbent layers 17, 17′.

In addition or alternatively, it may be that one or two, or more, or all, channels 26 of one absorbent layer 17 do not superpose the channels 26′ of the adjacent absorbent layer 17′. Said channels 26 of one layer 17 may be, for example, complementary with the channels 26 of the adjacent layer 17′. By complementary, it is meant that the channels 26′ of the second absorbent layer form an extension of the channels 26 of the first absorbent layer 17.

Likewise, the acquisition-distribution system or layer 9, or another layer of the article, may also comprise channels, which may or may not correspond to the channels 26 of the absorbent core 7.

The one or more adhesive materials may be at least present in the channels 26, 26′, or part thereof, and the supporting sheets 16, 16′ may be adhered to one another in said channels 26, 26′ by one or more of these adhesive materials. Adhesive may be present between the second supporting sheet 16′ and the acquisition material layer, or between the acquisition material layer and the absorbent layer 17 to improve adhesion of both layers.

In any of these cases, the acquisition material layer may fold into (i.e. undulate into) said channels 26 or part thereof. It may be adhered to the supporting sheet 16 in said channels 26, e.g. by an adhesive material, as described herein. Alternatively, or in addition, it may be adhered to the walls of the channels 26 and/or 26′ or part thereof.

As the core absorbs more liquid, the depressions formed by the channels of the present invention will become deeper and more apparent to the eye and the touch from the exterior of the article as the backsheet is pushed outwardly by the expending absorbent material.

### Absorbent Material

Absorbent material 50 in the core may comprise superabsorbent polymer material (“SAP”), optionally combined with cellulosic material (including for example cellulose, comminuted wood pulp in the form of fibers).

In some embodiments, the absorbent material 50 may comprise at least 60%, or at least 70% by weight of superabsorbent polymer material, and at the most 40% or at the most 30% of cellulosic material. For absorbent cores comprising a relatively high proportion of superabsorbent polymer enclosed within the core wrap, the SAP content may represent in particular at least 85%, 90%, 95% and up to 100%, of superabsorbent polymer by weight of the absorbent material. The absorbent material may in particular comprise no or only small amount of cellulose fibers, such as less than 20%, in particular less than 10%, 5% or even 0% of cellulose fibers by weight of the absorbent material. The absorbent material may thus advantageously consist or consist essentially of SAP. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP used may have a CRC value of more than 20 g/g, more than 24 g/g, from 20 to 50 g/g, from 20 to 40 g/g, or from 24 to 30 g/g, specifically reciting all 0.1 g/g increments within the above-specified ranges and any ranges created therein or thereby. The SAP useful with the present disclosure may include a variety of water-insoluble, but water-swellable polymers capable of absorbing large quantities of fluids.

The SAP may be typically in particulate forms (superabsorbent polymer particles), but it not excluded that other forms of SAP may be used such as a superabsorbent polymer foam for example. The absorbent core may thus be relatively thin, in particular thinner than conventional cores comprising cellulosic fibers.

The absorbent polymer particles can be selected among polyacrylates and polyacrylate based materials that are internally and/or surface cross-linked, such as for example partially neutralized cross-linked polyacrylates or acid polyacrylate. Examples of absorbent polymer particles suitable in the present disclosure are described for instance in the PCT Patent Application WO 07/047598, WO 07/046052, WO2009/155265 and WO2009/155264. Particulate absorbent polymer materials may be made of poly(meth)acrylic acid polymers. However, starch-based particulate absorbent polymer material may also be used, as well as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

The SAP may be of numerous shapes. The term “particles” refers to granules, fibers, flakes, spheres, powders, platelets and other shapes and forms known to persons skilled in the art of superabsorbent polymer particles. The SAP particles may be in the shape of fibers, i.e., elongated, acicular superabsorbent polymer particles. The fibers may also be in the form of a long filament that may be woven. SAP may be spherical-like particles. The absorbent core may comprise one or more types of SAP.

For most absorbent articles, liquid discharges from a wearer occur predominately in the front half of the absorbent article, in particular for a diaper. The front half of the article (as defined by the region between the front edge and a transversal axis Y) may therefore comprise most of the absorbent capacity of the core. Thus, at least 60% of the SAP, or at least 65%, 70%, 75%, 80%, or 85% of the SAP may be present in the front half of the absorbent article, while the remaining SAP may be disposed in the rear half of the absorbent article. Alternatively, the SAP distribution may be uniform through the core or may have other suitable distributions.

The absorbent material 50 defines an absorbent material deposition area 11, as seen from above within the plane of the core 7 as shown on FIG. 2. The deposition area 11 comprises the channels 26 encompassed within. The absorbent material deposition area 11 can be generally rectangular, for example as shown in FIG. 2, but other shapes can also be used such as a “T” or “Y” or “sand-hour” or “dog-bone” shape. In particular the deposition area may show a tapering along its width at the crotch region of the core. In this way, the absorbent material deposition area 11 may have a relatively narrow width in an area of the core 7 intended to be placed in the crotch region of the absorbent article. This may provide for example better wearing comfort.

The total amount of SAP present in the absorbent core may also vary according to expected user. Diapers for newborns may require less SAP than infant, child, or adult incontinence diapers. The amount of SAP in the core may be about 5 to 60 g or from 5 to 50 g, specifically reciting all 0.1 increments within the specified ranges and any ranged formed therein or thereby. The average SAP basis weight within the (or “at least one”, if several are present) deposition area 11 of the SAP may be at least 50, 100, 200, 300, 400, 500 g/m² or more. The areas of the channels (e.g., 26, 26′, 27, 27′) present in the absorbent material deposition area 11 are deducted from the absorbent material deposition area to calculate this average basis weight.

### Supporting Sheet(s) and Core Wrap

The supporting sheet 16 may surround the absorbent material to form the core wrap 10, or two supporting sheets 16, 16′ may be joined to form the core wrap 10. In one embodiment, the core wrap 10 may comprise a top layer generally forming the top side of the core 7 and a bottom layer generally forming the bottom side of the core 7. The top and bottom layers may be formed by two separate substrates (e.g., the supporting sheets 16, 16′) which may be the same or different material (the top layer being for example hydrophillically treated). However, any other known core wrap constructions may also be used, for example wherein the core wrap is formed of a single web wrapped around the absorbent material with one single longitudinal seal.

The top and bottom layer can be attached by gluing or otherwise to form at least one C-wrap seal along each of the longitudinally-extending side edges of the core. The material of the top and bottom layers may be a nonwoven web, such as a laminate comprising spunbond (“S”) or meltblown (“M”) layer. For example spunmelt polypropylene nonwovens are suitable, in particular those having a laminate web SMS, or SMMS, or SSMMS, structure, and having a basis weight range of about 5 gsm to 15 gsm. Suitable materials are for example disclosed in U.S. Pat. No. 7,744,576, US 2011/0268932 A1, US 2011/0319848 A1 and US 2011/0250413 A1. The bottom layer may be inherently hydrophobic but air-permeable, and the top layer may be hydrophillically treated. There may be a seal along the front edge and back edge of the core wrap 11 for better containment of the absorbent material 50 but many cores do not have such transversal seals.

The supporting sheets 16 and/or core wrap layers may have a CD-extensibility or a MD-extensibility.

In one of the embodiment herein, the supporting sheet 16 and/or a core wrap layer has undulations that fold (undulate) into said first and second channels 26, and optionally in to said further channels, of part thereof. For example the undulations may extend over about the full longitudinal dimension of the channel; they may for example extend to complete average height of the absorbent layer 17 and/or channel 26, or for example only up to 75% thereof, or up to 50% of the average height of the absorbent layer 17/channel 26. This aids immobilization of the absorbent material 50 adjacent said channels 26 and said channels 26 of said layers. In order to form easily said undulations, the supporting sheet 16, and/or core wrap layer, may have a basis weight that is less than 60 gsm, or for example less than 50 gsm, for example from 5 gsm to 40 gsm, or to 30 gsm.

The undulations may be adhered with said one or more adhesive material, e.g. said second adhesive material, to said walls of said channels 26. The top layer (e.g., the supporting sheet 16) may alternatively, or in addition, be attached to said bottom layer (e.g., second supporting sheet 16′) or another other layer of the article through the channels 26, for example with the first and/or the second adhesive material.

### Adhesive Material

The adhesive material(s) may not only help in immobilizing the absorbent material on the supporting sheet but may also help in maintaining the integrity of channels in the absorbent layer absorbent core during storage and/or during use of the disposable article. The adhesive material may help to avoid a significant amount of absorbent material migrating into the channels. Furthermore, when the adhesive material is applied in the channels or on the supporting sheet portions coinciding with the channels, it may thereby help to adhere the supporting sheet of the absorbent layer to said walls, and/or to another layer of the article.

The first adhesive material 40 may comprise a thermoplastic adhesive material. The thermoplastic adhesive material may be applied as a continuous layer (i.e. uniformly) over the absorbent layer. In some embodiments, the thermoplastic adhesive material contacts the absorbent polymer particles (and cellulose when present) and part of the substrate layer when the absorbent polymer particles (and cellulose when present) are deposited in clusters.

In some embodiments, the thermoplastic adhesive material may be applied as a fibrous layer forming a fibrous network over the absorbent layer. The thermoplastic adhesive fibrous layer may be at least partially in contact with the absorbent polymer particles (and cellulose when present) and partially in contact with the supporting sheet when the absorbent polymer particles (and cellulose when present) are deposited in clusters. Thereby, the thermoplastic adhesive material may provide cavities to cover the absorbent polymer particles, and thereby immobilizes this material (and the channels when present).

Thermoplastic adhesive materials suitable for immobilizing the absorbent polymer particles typically combine good cohesion and good adhesion behavior. Good adhesion promotes good contact between the thermoplastic adhesive material and the absorbent polymer particles and the substrate layer. Good cohesion reduces the likelihood that the adhesive breaks, in particular in response to external forces, and namely in response to strain. When the absorbent core absorbs liquid, the absorbent polymer particles swell and subject the thermoplastic adhesive material to external forces. The thermoplastic adhesive material may allow for such swelling, without breaking and without imparting too many compressive forces, which would restrain the absorbent polymer particles from swelling.

Thermoplastic adhesive materials suitable for use in the present disclosure includes hot melt adhesives comprising at least a thermoplastic polymer in combination with a plasticizer and other thermoplastic diluents such as tackifying resins and additives such as antioxidants. Exemplary suitable hot melt adhesive materials 40, 60 are described in EP 1447067 A2. In some embodiments, the thermoplastic polymer has a molecular weight (Mw) of more than 10,000 and a glass transition temperature (Tg) below room temperature or −6° C.>Tg<16° C. In certain embodiments, the concentrations of the polymer in a hot melt are in the range of about 20 to about 40% by weight. In certain embodiments, thermoplastic polymers may be water insensitive. Exemplary polymers are (styrenic) block copolymers including A-B-A triblock structures, A-B diblock structures and (A-B)n radial block copolymer structures wherein the A blocks are non-elastomeric polymer blocks, typically comprising polystyrene, and the B blocks are unsaturated conjugated diene or (partly) hydrogenated versions of such. The B block is typically isoprene, butadiene, ethylene/butylene (hydrogenated butadiene), ethylene/propylene (hydrogenated isoprene), and mixtures thereof.

Other suitable thermoplastic polymers that may be employed are metallocene polyolefins, which are ethylene polymers prepared using single-site or metallocene catalysts. Therein, at least one comonomer can be polymerized with ethylene to make a copolymer, terpolymer or higher order polymer. Also applicable are amorphous polyolefins or amorphous polyalphaolefins (APAO) which are homopolymers, copolymers or terpolymers of C2 to C8 alpha olefins.

The thermoplastic adhesive material, typically a hot-melt adhesive material, is generally present in the form of fibers, i.e. the hot melt adhesive can be fiberized. In some embodiments, the thermoplastic adhesive material forms a fibrous network over the absorbent polymer particles. Typically, the fibers can have an average thickness from about 1 μm to about 100 μm, or from about 25 μm to about 75 μm, and an average length from about 5 mm to about 50 cm. In particular, the layer of hot melt adhesive material can be provided such as to comprise a net-like structure. In certain embodiments, the thermoplastic adhesive material is applied at an amount of from 0.5 to 30 g/m², or from 1 to 15 g/m², or from 1 and 10 g/m² or even from 1.5 and 5 g/m² per supporting sheet 16.

A typical parameter for an adhesive suitable for use in the present disclosure can be a loss angle tan Delta at 60° C. of below the value of 1, or below the value of 0.5. The loss angle tan Delta at 60° C. is correlated with the liquid character of an adhesive at elevated ambient temperatures. The lower tan Delta, the more an adhesive behaves like a solid rather than a liquid, i.e. the lower its tendency to flow or to migrate and the lower the tendency of an adhesive superstructure as described herein to deteriorate or even to collapse over time. This value is hence particularly important if the absorbent article is used in a hot climate.

It may be beneficial, e.g. for process reasons and/or performance reasons, that the thermoplastic adhesive material has a viscosity of between 800 and 4000 mPa·s, or from 1000 mPa·s or 1200 mPa·s or from 1600 mPa·s to 3200 mPa·s or to 3000 mPa·s or to 2800 mPa·s or to 2500 mPa·s, at 175° C., as measurable by ASTM D3236-88, using spindle 27, 20 pmp, 20 minutes preheating at the temperature, and stirring for 10 min.

The thermoplastic adhesive material may have a softening point of between 60° C. and 150° C., or between 75° C. and 135° C., or between 90° C. and 130° C., or between 100° C. and 115° C., as can be determined with ASTM E28-99 (Herzog method; using glycerine).

In one embodiment herein, the thermoplastic adhesive component may be hydrophilic, having a contact angle of less than 90°, or less than 80° or less than 75° or less than 70°, as measurable with ASTM D 5725-99.

In some embodiments, the absorbent layer may also comprise an adhesive material deposited on the substrate before application of the absorbent polymer particles on the substrate layer, referred to herein as the second adhesive 60. The second adhesive may enhance the immobilization of the absorbent polymer particles on the supporting sheet 16. The second adhesive material 60 may be a thermoplastic adhesive material, and comprise the same thermoplastic adhesive material as described hereinabove or it may be different. An example of commercially available adhesive is H.B. Fuller Co. (St. Paul, Minn.) Product No. HL-1620-B. The thermoplastic adhesive material may be applied to the supporting sheet by any suitable means.

### Signals

The absorbent articles of the present invention may comprise signals that communicate to a consumer or wearer the functionality and benefits of the absorbent core, including comfort, fit and/or absorbency. Non-limiting examples of such signals may include printed adhesive layers, backsheet graphics, embossing of the topsheet and/or acquisition layer, and combinations thereof. Signals may be particularly useful in indicating the presence of channels 26 and/or the functionality of channels 26.

Indicia may serve as a signal that may be used to communicate the existence and the benefits of the core 7 and/or the functionality of channels 26 (whether channels are present or not in the structure). “Indicia”, as used herein, may comprise one or more inks with pigments, adhesives with pigments, words, designs, trademarks, graphics, patterns, and/or pigmented areas, for example. The indicia may typically be a different color than: (1) the layer that it is printed on, positioned on, or applied to; or (2) a different color than other layers of an absorbent article. The phrase a “different color” means a different shade of the same color (e.g., dark blue and light blue) or may be completely different color (e.g., blue and red). The indicia should be at least partially visible from either a wearer facing surface, a garment facing surface, or both of an absorbent article, although the indicia may not be printed on, positioned or, on applied to the wearer or garment facing surfaces of the absorbent articles. The indicia may comprise a light activatable material, a liquid activatable material, a pH activatable material, a temperature activatable material, a menses activatable material, a urine activatable material, or BM activatable material, or an otherwise activatable material. These activatable materials may typically undergo a chemical reaction, or other reaction, to change the indicia from one color to a different color, from one color to a different shade of the same color, from a color that is not visually distinguishable in an absorbent article to a color that is visually distinguishable in an absorbent article, or from a color that is visually distinguishable in an absorbent article to a color that is not visually distinguishable in an absorbent article. In an instance, the indicia may grow or shrink or display a graphic/not display a graphic after the indicia undergoes the reaction. In other instances, the indicia may be activated by a stress or a strain during manufacture or wear. The indicia may be white or non-white. If the indicia is white in color, at least one layer may be non-white so that the indicia is visible from a wearer and/or garment facing surface of the absorbent articles, for example. The indicia may comprise embossments, fusion bonds, or other mechanical deformations. In some instances, the indicia may be formed within either a sheath or a core of bicomponent fibers. For example, a core may be white, while a sheath may be blue, or vice versa.

The indicia 600 may be on, positioned on, formed on, formed with, printed on, or applied to all of, or part of, a certain layer, such as a topsheet or an acquisition layer, for example. The indicia may also be on, positioned on, formed on, formed with, printed on, or applied to one or more layers, or on all suitable layers of an absorbent article. The indicia may be on, positioned on, formed on, formed with, printed on, or applied to either side, or both sides, of the one or more layers of an absorbent article. In some instances, suitable layers for indicia placement may comprise one or more of a topsheet, a secondary topsheet, an acquisition material, a distribution material, a core bag, a wearer-facing side of the core bag, a garment-facing side of the core bag, and/or an additional layer positioned at least partially intermediate the topsheet and the wearer-facing side of the core bag (hereafter “suitable layers for indicia placement”).

Either in addition to or separate from the indicia 600 described above, any one or more of the suitable layers for indicia placement, or a portion thereof, may have a color different than any one or more of the remaining layers for indicia placement, or a portion thereof. The definition of the phrase “different color” above also applies to this part of the disclosure. In some instances, the indicia may be a different color than any one or more of the suitable layers for indicia placement. Alternatively, the indicia may be on one of the suitable layers for indicia placement while another one of the remaining suitable layers for indicia placement may be a different color than the indicia. One example may be a blue indicia on a white acquisition layer with the topsheet being teal. In another example, a blue indicia may be on a white acquisition layer with the topsheet also being white. In both of such instances, the blue indicia may be viewable from a wearer-facing surface.

In some instances, a visible color of a portion of, or all of, the interior (wearer-facing surface) of an absorbent article may be coordinated with and/or compliment a visible color of a portion of, or all of, the exterior (garment-facing surface) of the absorbent article, as described in further detail in U.S. Pat. No. 8,936,584. The indicia visible from the interior may also be coordinated with and/or compliment the indicia visible from the exterior of the absorbent article. In still other instances, the visible indicia and/or color from the interior may also be coordinated with or compliment the indicia and/or color visible from the exterior of the absorbent article.

In addition to that described above, a first portion of one of the suitable layers for indicia placement may be a first color and a second portion of the same of the suitable layers for indicia placement may be a second color. The first and second colors may be a different color. In other instances, a first portion of one of the suitable layers for indicia placement may be a first color and a second portion of a different one of the suitable layers for indicia placement may be a second color. The first and second colors may be a different color.

In an instance, in an absorbent article, one of a topsheet, an acquisition material, a portion of a core bag, or an additional layer (e.g., a distribution layer) may be a different color than a different one of the topsheet, the acquisition material, the portion of the core bag, or the additional layer. In another instance, in an absorbent article, one of a portion of a topsheet, a portion of an acquisition material, a portion of a core bag, or a portion of an additional layer may be a different color than a different one of the portion of the topsheet, the portion of the acquisition material, the portion of the core bag, or the portion of the additional layer. In another instance, in an absorbent article, a first portion of one of a topsheet, an acquisition material, a core bag, or an additional layer may be a different color as a second portion of the same one of the topsheet, the acquisition material, the core bag, or the additional layer.

Indicia 600 may comprise a pigmented adhesive or ink that is applied to: (1) a liquid permeable substrate, when used as a topsheet, on a garment-facing side; (2) either side of an acquisition layer; (3) either side of a secondary topsheet; (3) either side of the core or core bag; or (4) either side of an additional layer positioned at least partially intermediate the topsheet and an absorbent core.

In some embodiments, the indicia 600 comprises a pattern 110. The pattern 110 may be visible through the topsheet. The pattern 110 may be formed from any suitable indicia. In one nonlimiting example, a printed adhesive layer adhesive layer 100 is applied in a pattern 110. In some embodiments, the pattern 110 is formed by a printed adhesive layer in combination with one or more other forms of indicia. The layer of printed adhesive 100 may be visible through the topsheet.

In some embodiments, a fluid, such as an adhesive, may be applied or printed onto an advancing substrate. The fluid application apparatus may include a slot die applicator and a substrate carrier. The slot die applicator may include a slot opening, a first lip, and a second lip, the slot opening located between the first lip and the second lip. And the substrate carrier may include one or more pattern elements and may be adapted to advance the substrate past the slot die applicator as the slot die applicator discharges adhesive onto the substrate. In operation, when the first surface of the substrate is disposed on the substrate carrier, the substrate carrier advances the second surface of the substrate past the slot opening of the slot die applicator. In turn, the substrate is intermittently compressed between the slot die applicator and the pattern surface of the pattern element. As the substrate is intermittently compressed, adhesive discharged from the slot die applicator is applied onto the second surface of the advancing substrate in an area having a shape that is substantially the same as a shape defined by the pattern surface. In some embodiments, the adhesive may be a different color than the substrate. The adhesive may comprise pigments or dyes. Other methods and apparatuses involved for the application of adhesives in pre-determined patterns to an advancing substrate are disclosed in U.S. Pat. No. 8,186,296. In some embodiments, the fluid printed on the substrate may be an ink without an adhesive, or both, or blends of adhesive and ink.

The pattern 110 may be applied to any suitable layer for indicia placement, including a supporting sheet, core wrap, or acquisition material layer.

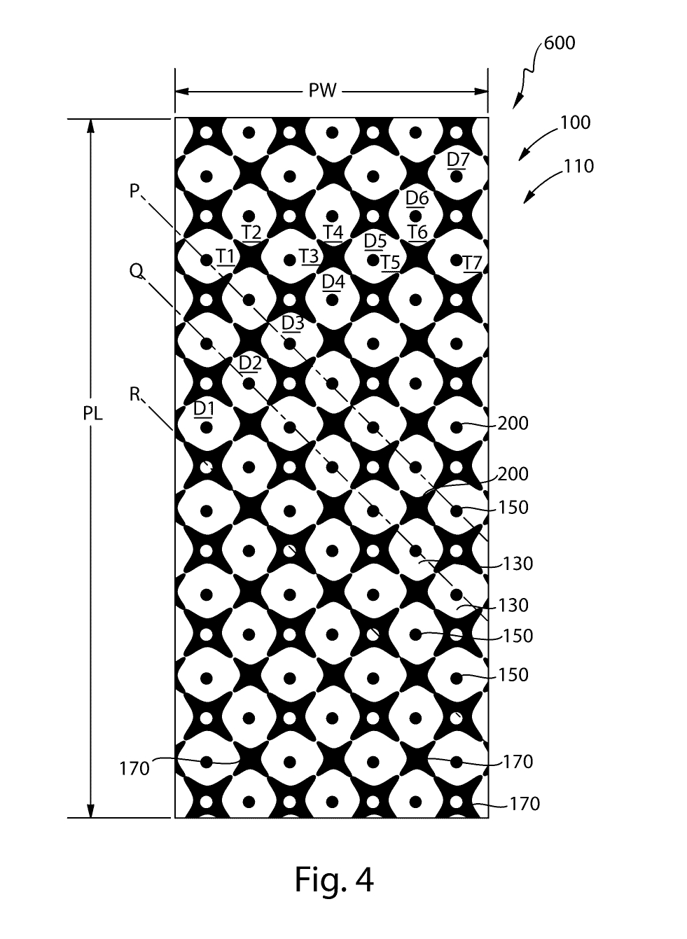

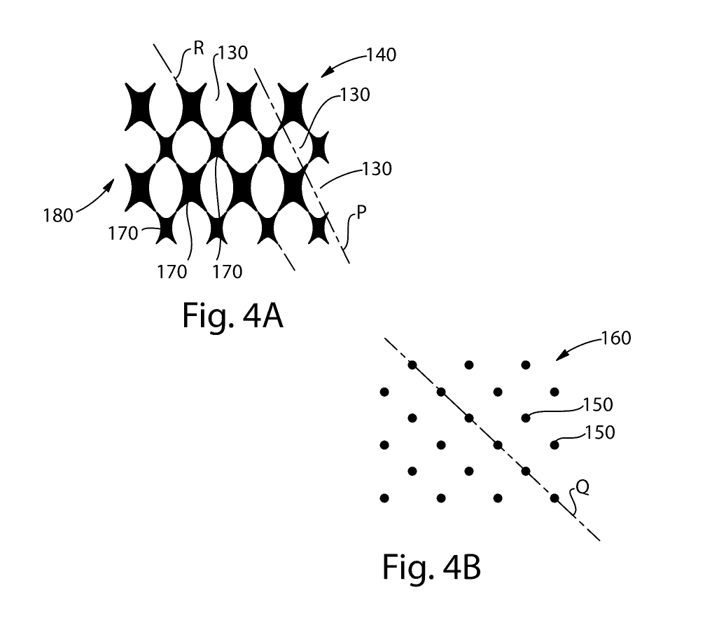

As shown in FIG. 4, the pattern 110 may comprise an average width PW in the transverse direction extending between two longitudinal edges and an average length PL in the longitudinal direction extending between two transverse edges. The transverse and longitudinal dimensions of the pattern may be parallel to the transverse and longitudinal dimensions of the absorbent layer 17 respectively. In one nonlimiting example, the pattern width and length are substantially the same as the width and length of the absorbent layer 17 respectively. In another nonlimiting example, the pattern's width is less than the average width of the layer and/or the pattern's length is less than the average length of the layer.

The pattern may comprise a first design element 130. The first design element 130 may repeat throughout the pattern 110 in the longitudinal and/or transverse dimensions. The first design element may be oriented differently at different areas in the pattern. In one nonlimiting example, the first design element repeats between about 5 and about 11 times, or between about 5 and about 9 times, or between about 5 and about 7 times in the transverse direction through the width, PW, as can be seen, for example, in FIG. 4. For clarity, the repetition is counted based on the number adjacent like elements across the transverse dimension. In other words, in FIG. 4, the repetition can be counted by the number of elements in two rows, as the elements in said two rows are adjacent, or adjacent elements disposed in a diagonal line. For example, the first element 130 repeats 7 times throughout the transverse direction in FIG. 4 as shown by the numbers T1-T7 (counting adjacent first elements in two rows) and the numbers D1-D7 (showing the count along the diagonal). In addition, an element cut off by the edge of the pattern will be included in the count if the majority of the element is present. It is believed that the repetition allows a caregiver or wearer to identify separate individual elements across the pattern, which enhances the pattern definition and aids in using the pattern to communicate intended benefits of the product (e.g., softness, absorbency, fit etc.). In addition, when the pattern 110 is created or partially created by a printed adhesive layer, repetition in this manner provides a sufficient amount of adhesive for suitable bonding while also providing a consumer-preferred contrast between the printed area and non-printed area of the pattern.

The repetition of the first design element 130 results in a primary motif 140 as illustrated in FIG. 4A. The primary motif may be disposed diagonally, as shown with line P. In this way, the overall pattern 110 may be provided as at least partially tiled and/or symmetrical, which resembles textiles such as quilts, pointelles and other fabrics. The textile-like pattern is believed to connote softness and/or a garment-like texture and/or appearance.

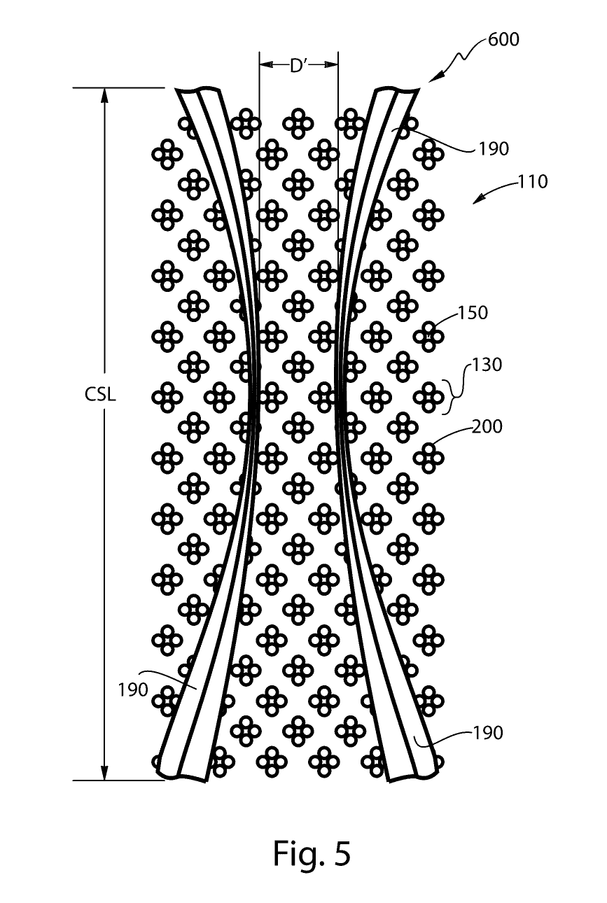

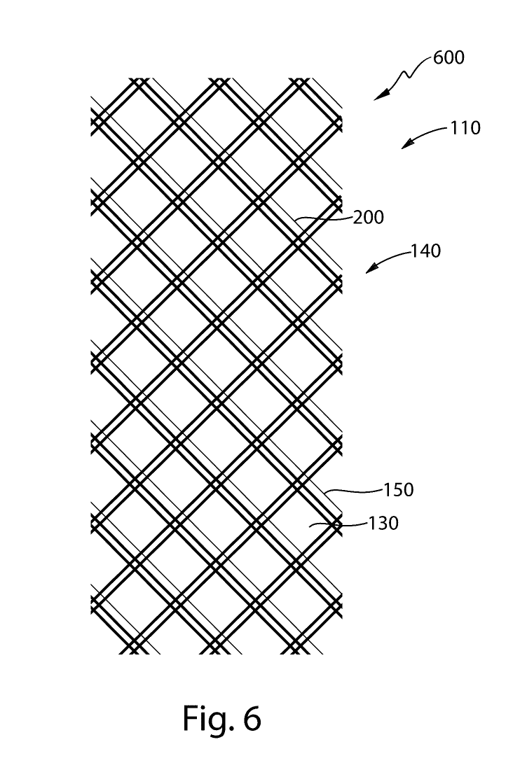

The first design element 130 may comprise any shape suitable for achieving the above-described purposes and/or process capability and communication balance in the pattern as discussed below. Nonlimiting examples of shapes from which the first design element may be selected include: a diamond, an oval, a circle, a square, a rectangle, a pod (i.e., a closed or semi-closed shape that comprises a combination of straight and curvilinear line segments in its perimeter), a quatrefoil, and combinations thereof. In some instances, the first design element may comprise a cluster of shapes as shown for example in FIG. 5. In some instances, the first design element may comprise a polygon or pod defined by intersecting lines (i.e., lines being straight or curvilinear) as illustrated in FIG. 6. In some such instances, the primary motif may comprise a grid of intersecting lines. In some further embodiments, the first design element comprises a shape that provides a textile-like appearance to the primary motif. In this way, caregivers and end users of the article may perceive the article or a portion of the article to be soft and/or garment-like. Nonlimiting examples of first elements that provide a textile-like appearance to the pattern include a diamonds, pods, quatrefoils, and/or shapes with curvilinear, wavy or stitch-like perimeters.

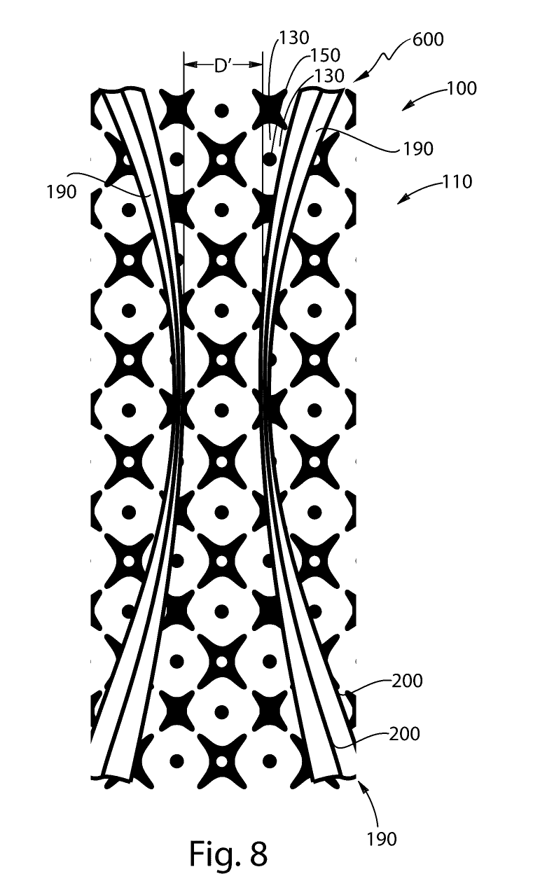

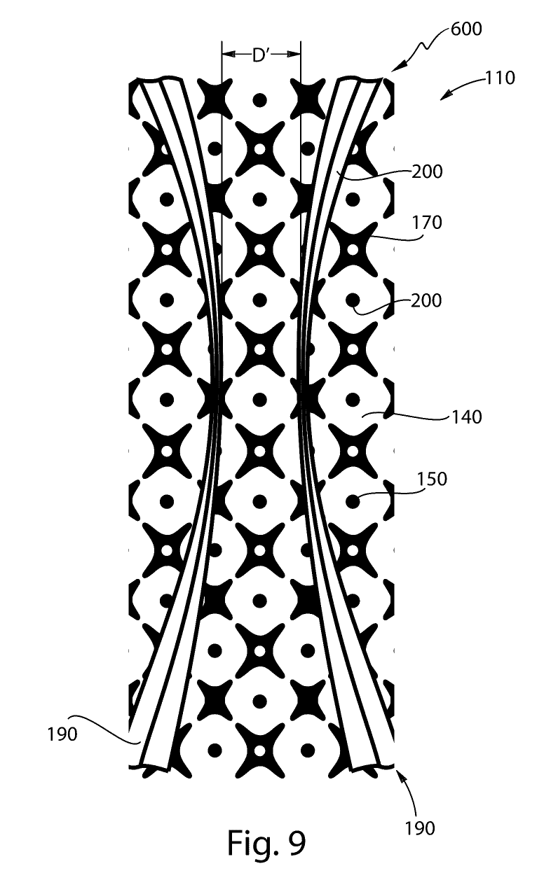

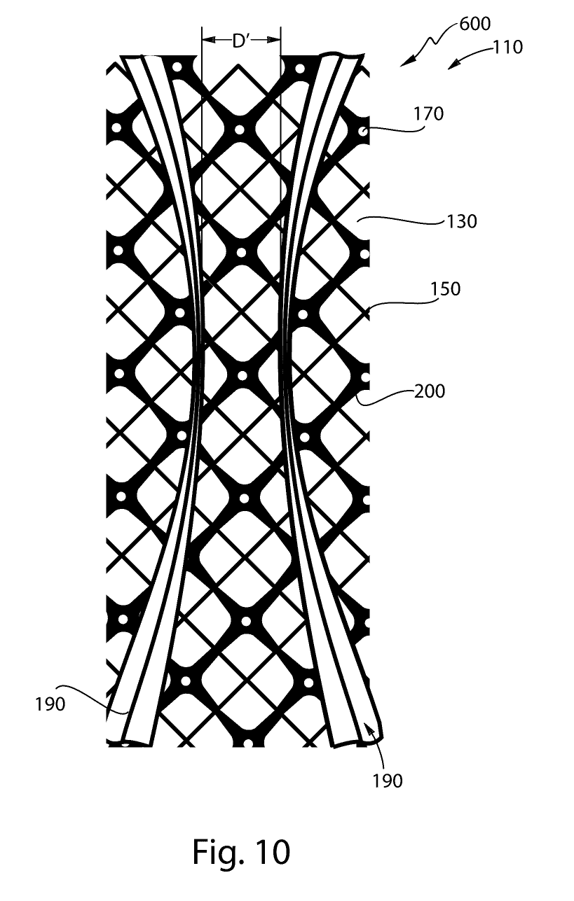

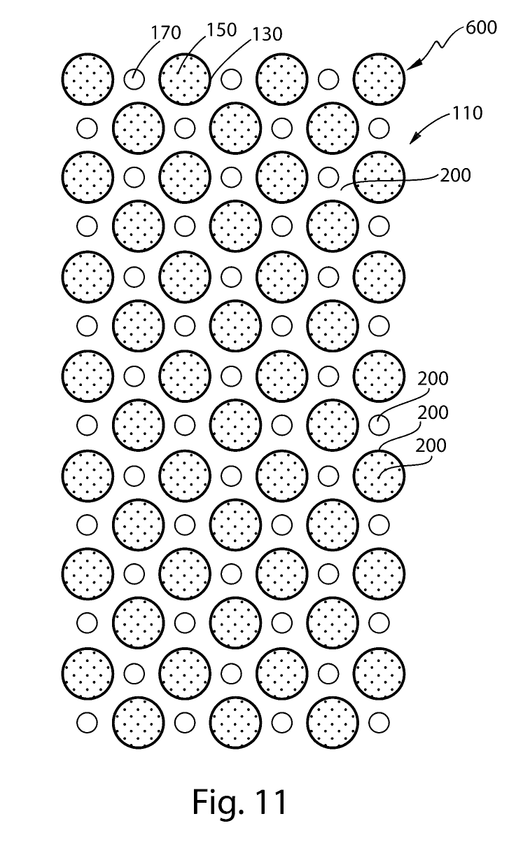

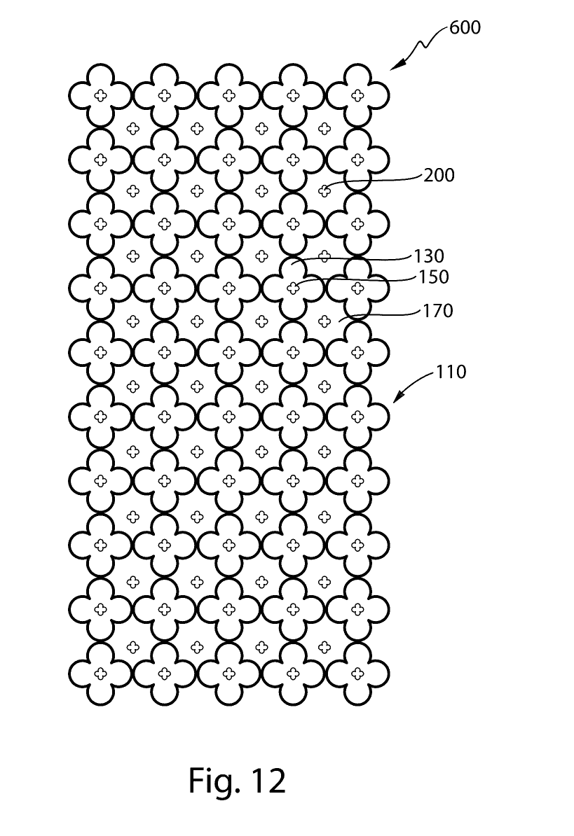

The pattern may further comprise a second design element 150 shown for example in FIGS. 4 and 4B. The second design element 150 may repeat throughout the pattern 110 in the longitudinal and/or transverse dimensions. The second design element may be oriented differently at different areas in the pattern. In one nonlimiting example, the second design element repeats between about 5 and about 11 times, between about 5 and about 9 times, or between about 5 and about 7 times in the transverse direction through the width, PW, as can be seen, for example, in FIG. 4. The second design element may complement the first design element. In one nonlimiting example, at least a portion of one second design element is disposed within one of the first design elements as shown in FIGS. 4-12. In a further nonlimiting example, a second design element is disposed in the center of a first design element. By disposing the second design element within the first design element, a three-dimensional appearance and/or shadowing can be created. Said three-dimensionality and/or shadowing connotes depth which may be perceived as softness. In addition, the inventors have observed that the three-dimensional appearance connotes that the article is absorbent, in particular, that fluid will flow down into the core 7.

The second design element 150 may repeat to form a secondary motif 160. The secondary motif may be disposed diagonally, as illustrated for example in FIGS. 4 and 4B with line Q. In this way, the secondary motif may work in conjunction with the primary motif to provide a textile-like pattern having a three-dimensional appearance.

The second design element may comprise any shape suitable for achieving the above-described purposes, complementing the first design element in such a way as to provide a three-dimensional appearance and/or achieving process capability and communication balance in the pattern as discussed below. Generally, the second design element may be smaller and/or different in color and/or intensity than the first design element. The second design element may differ from the first design element by one of: line thickness, color, shape, size and combinations thereof. Nonlimiting examples of shapes from which the second design element may be selected include: a diamond, an oval, a circle, a square, a rectangle, a pod, a quatrefoil, a line and combinations thereof. In some instances, the second design element may comprise a cluster of shapes. In some instances, the second design element may comprise a polygon or pod defined by intersecting linear elements. In some such instances, the secondary motif may comprise a second grid of intersecting lines (i.e., straight or curvilinear lines). Where the primary and secondary motifs comprise grids of intersecting lines, said grids may be offset such that a portion of the secondary element (e.g., a line) is visible inside a first design element. In further embodiments, the secondary motif may comprise a series of parallel lines disposed diagonally. Said parallel lines may mirror a portion of the primary motif as shown in FIG. 6. In some further embodiments, the second design element comprises a shape that provides a textile-like appearance to the secondary motif. In this way, caregivers and end users of the article may perceive the article or a portion of the article to be soft and/or garment-like. Nonlimiting examples of second design elements that provide a textile-like appearance to the pattern include diamonds, pods, quatrefoils, and/or shapes with curvilinear, wavy and/or stitch-like perimeters.

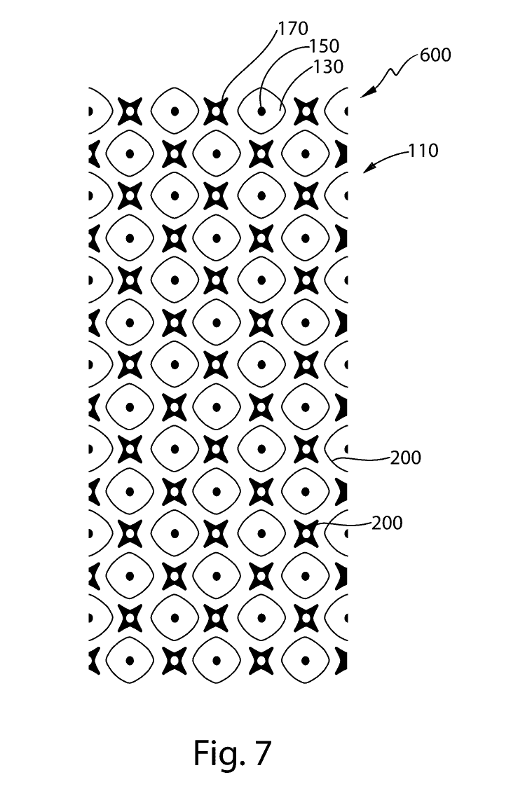

In some embodiments, the pattern comprises a third design element 170, which may complement the first design element and/or the second design element to enhance the three-dimensional and/or textile appearance of the pattern. The third design element 170 may repeat throughout the pattern 110 in the longitudinal and/or transverse dimensions. The third design element may be oriented differently at different areas in the pattern. In one nonlimiting example, the third design element repeats between about 2 and about 11 times, or about 2 to about 9 times, or between about 3 and about 7 times in the transverse direction through the width, PW, as can be seen, for example, in FIG. 4. The third design element may complement the first and/or second design elements. In one nonlimiting example, at least a portion of one third design element is disposed between first design elements and/or in line segments or shapes forming the perimeter of first design elements as illustrated for example in FIG. 7. In a further nonlimiting example, the third design element may be a shape which repeats to form the perimeter of a first element as shown in FIG. 4. In this way, a three-dimensional appearance and/or shading can be created or enhanced.

The third design element 170 may repeat to form a third motif 180. The third motif may be disposed diagonally, as illustrated for example in FIG. 4 with line R. In this way, the third motif may work in conjunction with the primary and/or secondary motifs to provide a textile-like pattern having a three-dimensional appearance. The third design element may comprise any shape suitable for achieving the above-described purposes, complementing the first or second design elements in such a way as to provide a three-dimensional appearance efficient and/or achieving process capability and communication balance in the pattern as discussed below. Generally, the third design element may be smaller and/or different in color or intensity than the first design element. The third design element may differ from the first and/or the second design element by one of: line thickness, color, shape, size and combinations thereof. Nonlimiting examples of shapes from which the third design element may be selected include: a diamond, an oval, a circle, a square, a rectangle, a pod, a quatrefoil, a line and combinations thereof. In some instances, the third design element may comprise a cluster of shapes.

The third motif 180 may be the inverse of the first motif as shown in FIG. 4A in some embodiments. Likewise, any of the motifs may be comprised of one or more elements that form a portion of more than one motif.

The indicia may comprise additional design elements in various embodiments. Nonlimiting examples of suitable patterns are illustrated FIGS. 4-12.

In some embodiments, the pattern 110 further comprises channel elements 190 as depicted in FIGS. 5, 8-10. The channel elements may comprise pairs of channel shapes (e.g., straight or contoured). The channel elements 190 may extend longitudinally, or substantially longitudinally (i.e., they may extend more in the longitudinal direction of the pattern than in the transverse direction). In one nonlimiting example, the channel elements 190 extend to one of the transverse edges of the pattern or from one transverse edge to the opposite transverse edge. In another nonlimiting examples, the channel elements 190 do not extend to either transverse edge. In further nonlimiting examples, the channel elements comprise an average length, CSL, which may be up to 90% of the average pattern length, PL, or up to 80% of the average pattern length, PL, or up to 75% of the average pattern length, PL. In one nonlimiting example, the channel element length, CSL, may correspond to the length of one or more channels in the absorbent layer 17. Where the channel or channel element is not parallel to the longitudinal axis, the length L′ of the channel or the length of the channel element CSL respectively is the length as measured by projection against the longitudinal axis. The lines forming the channel shapes may be continuous or substantially continuous. Each channel shape in a pair may be symmetric to its complement across the longitudinal axis X of the absorbent layer. Each channel shape may be symmetric at the center of the channel element's average width (i.e., the top and bottom of one line may be symmetric about the center of the channel element's width). In some embodiments, the channel elements appear to cover the remaining elements of the pattern as shown in FIGS. 5, 8-10. In this way, the channel elements appear to sit on top of the remaining pattern elements, adding to the three-dimensional perception and/or signaling that fluid will quickly be disposed in the channels and transported such that it will not excessively collect in any one area.

One or more design elements may be formed by any form of indicia, including adhesive with ink, ink (without adhesive), or the absence of ink (e.g., the non-printed area surrounding a printed area may form a design element). One or more design elements may be formed by an outline created from adjacent and/or abutting other design elements. The first, second and/or third design elements may repeat at different frequencies or at the same frequency.

In addition, the pattern 110 may comprise a color. In one embodiment, the color is blue 200 (i.e., any of the range of colors between green and purple on a visible color spectrum, but including shades of blue-violet). In one nonlimiting example, the color is light blue. “Light blue” as used herein refers to a color having L*a*b* values of: L*=from about 60 to about 90; a*=from about −48 to about −10; and b* from =about −38 to about −8 as determined by the Color Measurement Test Method herein, reciting for each range every 0.01 interval therein.

It is believed that blue connotes absorbency. Moreover, light blue may connote absorbency more effectively as the color will less likely be conveyed as a decorative pattern but will be perceived more as a signal of the article's performance. The indicia and/or pattern may comprise one or more shades of blue (i.e., blues have different L*a*b* values). Variations in the shades of blue may facilitate perceived three-dimensionality in the pattern. Any one or more of the design elements 130, 150, 170, 190 may comprise blue and/or light blue. Design elements may comprise the same shades of blue as used in other design elements. Design elements may comprise different shades of blue than the shade(s) used in other design elements. In some embodiments, an entire motif (e.g., the primary motif) or multiple motifs consist essentially of blue and/or light blue. In this way, the color is more evenly distributed across the pattern, signaling better distribution and absorption throughout the absorbent layer or core and/or provides a more apparent contrast against the color of the surface on which the motif is printed which enhances visibility. One or more channel signals may comprise or consist essentially of blue and/or light blue. In this way, the channel signals will signal faster absorption and elimination of wetness in any one particular area, and/or the channel elements are more visibly contrasted against the color of the surface on which said elements are printed.

In some embodiments, the pattern is printed on a white surface.

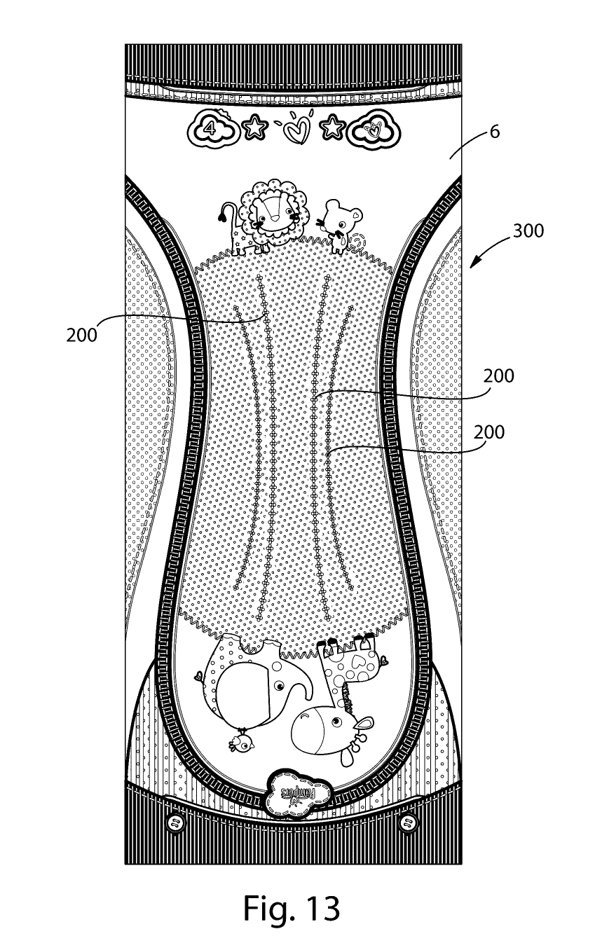

In some embodiments, the absorbent article may include indicia comprising graphics printed onto the backsheet as shown in FIG. 13. In an absorbent core having channels, fluid such as exudates will cause the absorbent material will expand in particular towards the backsheet, but the channels (which do not comprise absorbent material) will not expand. As such, the channels will become more perceptible through the backsheet as fluid is absorbed as they may form depressions. These depressions will become more pronounced as the absorbent article absorbs the fluid. The depth of these depressions formed by the channels on the backsheet side will be proportional to the amount of fluid absorbed. The visual appearance to the caregiver may be improved by providing a backsheet printing 300 which matches or at least indicates the region of the channels to the caregiver. Thus, the backsheet printing may comprise a line or a curve which substantially matches the shape and/or position of the channels. Said line or curve may comprise blue and/or light blue.

In some embodiments, the article may comprise both a pattern 110 visible through the topsheet and backsheet graphics 300, the combination of which work together or separately to accentuate certain aspects of the article, in particular absorbency features of the absorbent core and/or fit of the article (as may be aided by the presence of channels). In one nonlimiting example, the backsheet graphic and the pattern comprise blue and/or light blue. The backsheet graphics and the pattern comprise the same shades of blue. The backsheet graphics and the pattern comprise different shades of blue.

Further, in some embodiments, the graphics 300 visually signaling channels will not extend beyond the area of the channels 26 themselves, and/or the area where the absorbent core 7 is present.

In addition, the article may comprise other or additional indicia that signal the absorbent core features such as channels. One indicia may comprise embossing. Embossing may be done on the topsheet or on the absorbent core, or, in some embodiments, there may be multi-layer embossing of both the topsheet and the absorbent core. For example, thermal embossing may be done over channels (where there is no superabsorbent polymer) at a small gap setting to enhance the visual presence. Any embossing may be done alone or in combination with printing to help signal the absorbent core features, including channels. Further, any embossing may be done to the topsheet, core, or both, on-line (during assembly of the absorbent article's component parts) or prior to such assembly, e.g. at the site where a supplier of component parts makes the component part itself.

In some embodiments, the pattern 110 (e.g., a printed adhesive layer 100 comprising a pattern 110) and/or the backsheet graphics 300 may cover the majority or even all of the absorbent core. In other embodiments, the pattern or backsheet graphics may be provided only in discrete areas. For example, in some embodiments, the pattern or backsheet graphics may be provided so as to not overlap or intersect with the absorbent core channels 26. The term “overlap” as used herein means to cover over a part of, or to have an area in common. The term “intersect” as used herein means items that cut across or through each other, or that narrow and merge.

In some embodiments, backsheet graphics 300 are registered with the pattern 110 (or one or more motif or element within the pattern) and/or one or more article components (e.g., channel). In further embodiments, the pattern 110 (or one or more motifs or elements within the pattern) is registered with one or more backsheet graphics and/or article components. Any suitable means of registration known in the art may be used.

In some embodiments, any indicia 600, whether a pattern 110 visible through the topsheet, a backsheet graphic 300, or some combination, may match, or substantially match the shape or contour of absorbent core channels 26. In some embodiments, the indicia will not necessarily match or overlap or correlate with the shape or contour of the absorbent core channels 26, but will otherwise communicate or signal to the consumer the existence and/or benefits of the channels and/or absorbency, comfort and/or fit of the absorbent article.

### Process Capability and Successful Communication

There are difficulties in attempting to find combinations of elements that would provide sufficient visual appeal to caregivers with the technical and cost feasibilities of mass-producing absorbent articles. The distance between the design elements (e.g., innermost lines in the channel element, D′) as well as the distance between actual channels, D, may be important in providing absorbent cores that exhibit excellent process capability, technical product performance, and have a successful consumer communication. For example, if the distance between the channel element lines, D′, is too small, the process capability may be negatively impacted by equipment wear and material wrapping. Further, product performance may be negatively impacted by slower acquisition speeds when channels are too close together. If the distance between the channel element lines, D′, and/or the distance between lines of any element, is too large, the process capability may be negatively impacted by, for example, not having enough contact with the substrate to be printed on to remove ink and/or adhesive, thereby causing contamination to the printing equipment. Further, consumer communication may be negatively impacted by a pattern that is not consistently visible and having a low color intensity.

Further, in some embodiments, the adhesive coverage area may be important in providing absorbent cores that exhibit excellent process capability and technical product performance, and have a successful consumer communication. If the adhesive coverage area is too small, the process capability may be negatively impacted by not having enough glue coverage to maintain control of the patch in process. Further, in embodiments where the pattern 110 is at least partially formed by pigmented adhesive, consumer communication may be negatively impacted by not having enough adhesive pattern to deliver a consumer message. If the adhesive coverage area is too great, the process capability may be negatively impacted by having too much adhesive to deliver the process consistently and/or providing too much adhesive for proper fluid handling in the article. Further, consumer communication may be negatively impacted because of a lack of absorbency signaling (e.g., the pattern is too cluttered and/or fails to provide enough contrast between design elements).

Designs with more than 25 mm of nonprinted area within the pattern have been found to lack technically feasible because they are difficult to achieve from a manufacturing standpoint. Likewise, it may not be technically feasible to efficiently mass produce articles have less than 15% adhesive coverage in the pattern (where the pattern is formed by an adhesive layer), as such design may not provide enough adhesive to hold the absorbent article components together and may also be difficult to manufacture due to centerline difficulties. On the other hand, printed adhesive patterns comprising more than 30% adhesive coverage may provide too much adhesive for proper fluid handling and also may be difficult to manufacture due to centerline difficulties.

Combinations such as those shown in FIGS. 5, 8-10, are found to be visually successful and technically feasible, particularly when a printed adhesive layer 100 comprises the pattern 110. For example, these designs may be perceived as balancing the placement and amount of adhesive which allows for sufficient bonding of absorbent article layer components to each other, and also allows for good fluid handling and process-ability. These designs may be perceived as providing a suitable amount of space in between the element lines (e.g., including lines of channel shapes, portions of adjacent elements) which is able to be achieved from a manufacturing standpoint (e.g. a spacing of about 20 mm). Overall, these designs may suitably provide enough adhesive to hold the absorbent article components together, as pattern coverage with the adhesive is from about 15% to about 30%, and may be suitable to manufacture due to where process centerline have been established for base conditions to deliver at about 150N of force (nozzle to substrate+plate).

As such, in some embodiments of the present invention, the distance between adjacent portions of pattern elements, including innermost lines of the channel shapes in the channel element pair, is from about 2 mm to about 30 mm, or from about 5 mm to about 25 mm, or less than about 30 mm, or less than about 25 mm, or less than about 20 mm, or less than about 15 mm. In an embodiment, the distance between the channels may be substantially the same as the distance between the innermost lines of the channel element.

As such, in some embodiments of the present invention, the amount of adhesive comprising the printed adhesive layer is such that the pattern coverage with the adhesive is from about 10% to about 35%, from about 15% to about 30%, from about 20% to about 25%, less than about 35%, less than about 30%, more than about 10%, more than about 15%.

Processes for assembling the absorbent article 1 include conventional techniques known in the art for constructing and configuring disposable absorbent articles. For example, the backsheet and/or the topsheet can be joined to the absorbent core or to each other by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. under the designation HL-1258 or H-2031. While the topsheet, the backsheet, and the absorbent core may be assembled in a variety of well-known configurations, absorbent article configurations are described generally in absorbent article configurations are described generally in U.S. Pat. Nos. 5,554,145; 5,569,234; and 6,004,306.

### Color Measurement Test Method

All testing is performed in a room controlled at 23° C.±3 C.° and 50%±2% relative humidity. Samples are conditioned at 23° C.±3 C.° and 50%±2% relative humidity for 2 hours before testing.

Color is measured in accordance with ASTM E1349 using the CIE L*a*b* scale, 10° observer and D65 illuminant. Samples are prepared by removing the individual layers from the article with cryogenic spray. Care is taken to maintain orientation of the specimen during disassembly so that color is measured on the body facing surface of the specimen. Select the colored region of interest and place the specimen with a white backing material against the measuring port of the instrument. Record L*a*b* values to the nearest 0.01 units. Repeat this measure for a total of 5 replicate specimens, calculate the arithmetic mean for each L*a*b* value and report to the nearest 0.01 units.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be in the appended claims all such changes and modifications that are within the scope of this invention.

What is claimed is:

1. An absorbent article comprising:
a topsheet, a backsheet, an absorbent core disposed between the topsheet and the backsheet, a longitudinal central axis and a transverse central axis, and a pattern visible through the topsheet; wherein:
the pattern comprises:
a first design element that repeats to form a primary motif, and
a second design element that repeats to form a secondary motif; and
the pattern further comprises blue.

2. The absorbent article of claim 1 wherein the first and second design elements each comprise blue.

3. The absorbent article of claim 1, wherein the pattern comprises a channel element comprising a pair of channel shapes that run lengthwise in the machine direction of the absorbent article, each line being symmetric to its complement across longitudinal axis and at the center of its width; and wherein the distance between innermost lines of the channel shapes is less than about 25 mm.

4. The absorbent article of claim 1 wherein the primary and/or secondary motif is disposed diagonally with respect to the longitudinal axis.

5. The absorbent article of claim 1 wherein the pattern comprises a width, PW, in a transverse direction, and the first design element repeats about 5 to about 7 times through the width, PW.

6. The absorbent article of claim 1 wherein the primary motif comprises a textile-like appearance.

7. The absorbent article of claim 1 wherein at least a portion of one second design element is disposed in the center of first design element.

8. The absorbent article of claim 1 wherein the second design element is different than the first design element by one of the group consisting of line thickness, color, shape, size and combinations thereof.

9. The absorbent article of claim 1 wherein the pattern further comprises a third design element that repeats to form a third motif wherein the third design element is different from the first and/or the second design element by one of the group: line thickness, color, shape, size and combinations thereof.

10. The absorbent article of claim 1 further comprising a printed adhesive layer wherein the printed adhesive layer forms at least a portion of the pattern.

11. The absorbent article of claim 10, further comprising an acquisition layer disposed between the topsheet and the absorbent core, wherein the printed adhesive layer is printed onto the acquisition layer.

12. The absorbent article of claim 10, wherein the amount of adhesive comprising the printed adhesive layer is such that the pattern coverage with the adhesive is from about 15% to about 30%.

13. An absorbent article comprising:
a topsheet, a backsheet, an absorbent core disposed between the topsheet and the backsheet, a longitudinal axis and a transverse axis, wherein the absorbent core comprises a pair of channels, symmetrically disposed about the longitudinal axis; and
a pattern visible through the topsheet, wherein the pattern comprises:
a first design element that repeats to form a primary motif, and
a second design element that repeats to form a secondary motif;
a channel element; and
blue.

14. The absorbent article of claim 13 wherein the first and/or the second pattern comprises blue.

15. The absorbent article of claim 13 wherein the primary and/or secondary motif element is disposed diagonally with respect to the longitudinal axis.

16. The absorbent article of claim 13 wherein the pattern comprises a width, PW, in a transverse direction, and the first design element repeats about 5 to about 7 times through the width, PW.

17. The absorbent article of claim 13 further comprising a printed adhesive layer disposed between the topsheet and the absorbent core, wherein the printed adhesive layer forms at least a portion of the pattern.

18. The absorbent article of claim 17 further comprising an acquisition layer disposed between the topsheet and the absorbent core, wherein the printed adhesive layer is printed onto the acquisition layer.

19. The absorbent article of claim 17, wherein the amount of adhesive comprising the printed adhesive layer is such that the pattern coverage with the adhesive is from about 15% to about 30%.

20. The absorbent article of claim 13, wherein the absorbent core channels are contoured, and wherein the channel element substantially matches the contour of the absorbent core channels.

21. The absorbent article of claim 13 wherein the distance between innermost lines of the channels is less than about 25 mm.
